(19) United States
(12) Patent Application Publication
(10) Pub. No.: US 2025/0170166 A1
(43) Pub. Date: May 29, 2025

(54) HYALURONIC ACID HYDROGEL FORMULATION COMPRISING PHYTOESTROGEN FOR THE TREATMENT OF VAGINAL ATROPHY AND VAGINAL REJUVENATION AND PRODUCTION METHOD THEREOF

(71) Applicant: VSY BIYOTEKNOLOJI VE ILAC SANAYI ANONIM SIRKETI, Istanbul (TR)

(72) Inventors: Faruk OYTUN, Istanbul (TR); Efe CAN, Istanbul (TR); Busra Gizem KAYA, Istanbul (TR)

(73) Assignee: VSY BIYOTEKNOLOJI VE ILAC SANAYI ANONIM SIRKETI, Istanbul (TR)

(21) Appl. No.: 18/839,857

(22) PCT Filed: Dec. 13, 2022
(86) PCT No.: PCT/TR2022/051491

§ 371 Date: Aug. 20, 2024

(30) Foreign Application Priority Data
Feb. 23, 2022 (TR) 2022/002479

(51) Int. Cl. A61K 31/728; A61K 9/06; A61K 31/05; A61K 31/197; A61K 31/352; A61K 31/355; A61K 31/366; A61K 31/4415; A61K 31/455; A61K 31/51; A61K 31/525; A61K 31/593

(52) U.S. Cl. CPC A61K 31/728; A61K 9/06; A61K 31/05; A61K 31/197; A61K 31/352; A61K 31/355; A61K 31/366; A61K 31/4415; A61K 31/455; A61K 31/51; A61K 31/525; A61K 31/593

(57) ABSTRACT

A hyaluronic acid hydrogel formulation including phytoestrogen for the treatment of vaginal atrophy and vaginal rejuvenation and production method thereof is provided. The phytoestrogens included in the hyaluronic acid hydrogel formulation are herbal substances that can produce estrogenic effects and have the same structure and functions as estradiol. In this way, risks such as heart attack, stroke, and blood clotting seen in alternative treatment methods including hormone are not encountered.

### FIELD OF THE INVENTION

The present invention relates to a hyaluronic acid hydrogel formulation comprising phytoestrogen for the treatment of vaginal atrophy and vaginal rejuvenation, and production method thereof.

### BACKGROUND OF THE INVENTION

The corpus luteum of the ovary secretes large amounts of estrogen after maturation at puberty. Estrogen levels for young women vary with the menstrual cycle but are usually at high levels. But at the onset of menopause, the estrogen level in the body suddenly drops. This can lead to uncomfortable symptoms called menopause syndrome, such as hot flashes, night sweats, insomnia, mood swings, vaginal dryness, and vaginal atrophy.

Vaginal atrophy is the thinning of the vaginal mucosa in women due to estrogen deficiency. It causes a decrease in fibroblast activity in the connective tissue called lamina propria under the vaginal mucosa. It also causes a decrease in fibrils such as collagen and hyaluronic acid in the vaginal wall. Vaginal atrophy is most common during menopause. The inability to release estrogen from the ovaries due to menopause not only prevents menstruation, but also causes a deficiency of estrogen wherever it is effective in the body. When the vaginal mucosa begins to thin, problems such as frequently recurring vaginal infections, urinary problems and difficulty in sexual intercourse occur. Common symptoms of vaginal atrophy are as follows:
Thinning of the vaginal mucosa
Narrowing and shortening of the vaginal canal
Loss of moisture due to vaginal dryness
Burning sensation in the vagina
Spotting after intercourse
Pain and difficulty in sexual intercourse
Pain and burning sensation while urinating
Frequent urinary tract infections
Urinary incontinence

Although every woman experience atrophy in the vagina to a certain extent, this is known to be a major problem for 25% of menopausal women. Women who have never given birth or who have given birth by cesarean section experience more apparent problems than women who have had a normal birth. Women who have a regular sexual life experience less distress because of increased mucous blood flow. In addition, women who smoke may have vaginal dryness and experience more resistance to treatment.

Replacing what is missing is the basis of treatment in vaginal atrophy. For this reason, suppositories, gels or creams known as topical estrogens are used. These products also provide the necessary lubricity and moisture. In addition, the decrease in estrogen can be directly controlled with hormone replacement therapy (HRT). HRT is divided into two, namely systemic and topical administration [1, 2].

Systemic HRT is administered via pills or patches. Estrogen hormone is included in the blood circulation and provides relief of systemic symptoms. Studies have shown that systemic hormone therapy can increase the risk of heart attack, stroke, and blood clots. It has also been reported that long-term use may cause endometrial hyperplasia and increase the risk of cancer [3].

Topical HRT is usually preferred when only topical symptoms such as vaginal atrophy are experienced. Estrogen released during topical application can be directly absorbed by the vaginal mucosa. Due to the low dosage of topical HRT administration, estrogen hormone penetrates the body in lesser amounts than systemic estrogen therapy. This reduces the side effects and risks associated with systemic estrogen therapy [4].

Another method used in vaginal atrophy is PRP (platelet rich plasma). It is a good reservoir for many growth factors such as platelet-derived growth factor (PDGF), epidermal growth factor (EGF), transforming growth factor beta 1 (TGF-β1), vascular endothelial growth factor (VEGF), basic fibroblast growth factor (b-FGF), hepatocyte growth factor (HGF) and insulin-like growth factor (IGF-I). PRP is applied directly to the mucosa in the vagina, which activates the fibroblasts in the mucosa and provides the production of new collagen and elastin.

Another method used in vaginal atrophy is the use of fractional CO₂ lasers. Fractional CO₂ laser is applied to the inner wall of the vagina with a special apparatus. In this application, the laser is used in the vagina for the same purpose as for facial skin rejuvenation. After the application, the regeneration of the vaginal mucosa and the increase of support tissues such as new collagen in the vaginal wall are ensured. These improvements increase the moisture in the vagina and the rejuvenation of the vaginal wall enables narrowing.

When administering local and systemic estrogen preparations, it is necessary to carefully consider the side effects and risks of complications of drugs associated with systemic adsorption. Excessive estrogen levels in postmenopausal women can increase the risks of heart disease, breast cancer, thromboembolic complications, and cerebrovascular disease [5]. Low-dose local estrogens should be preferred, especially if the treatment is administered to elderly patients.

However, despite the large number of positive data showing improved vaginal health and sexual function with estrogens and moisturizers, recent data from Mitchell et al. are controversial as they both show no benefit when compared to placebo [6].

Some studies have reported that laser procedures have potential complications such as scarring, infection, pain, and decreased sexual desire. Another disadvantage of CO2 laser treatment is that it is a relatively new treatment and there has not been much study on a long-term clinical follow-up so far. In addition, the fact that it is an expensive treatment is another disadvantage [7].

Various agents are used in vaginal drug delivery systems, such as creams, gels, tablets, capsules, ointments, and the like. However, these substances have some limitations such as leaking, low retention time and daily dose requirement [8, 9]. Recently, many studies have been carried out on hydrogels to overcome these problems [10].

Postmenopausal women using hormone replacement therapy (HRT) usually stop the treatment before they complete it. This may be due to the increased cancer risk of local estrogens, as well as breast swelling/tenderness, bleeding or spotting, and

bloating, which are side effects associated with HRT [11]. As a result, the use of compounds comprising non-hormonal substances such as phytoestrogens is attracting more attention as safer alternatives [12].

Phytoestrogens can act as both estrogen agonists and antagonists by binding to

estrogen receptors [13]. The use of phytoestrogens as a treatment for menopausal symptoms has yielded significant results, according to several studies [14, 15, 16]. In a study, it was observed that orally taken phytoestrogens had no effect on

vaginal symptoms [17]. Therefore, local treatments are expected to attract more attention on these symptoms. Phytoestrogens are divided into three main classes as isoflavones, lignans and coumestans.

Hyaluronic acid (HA) in the formulation of the invention, which is currently used in many facial fillers, is a natural polysaccharide from the glycosaminoglycan family and forms an important part of the extracellular matrix of the skin and cartilage, including the vaginal mucosa. HA has the capacity to hold water molecules 1000 times its weight and plays a key role due to its properties such as the formation and protection of extracellular swelling, increasing the water holding capacity in the vulva-vaginal area, moisturizing the skin in case of inflammation and maintaining the water balance. It is also widely effective in the treatment of skin diseases, since it preserves tissue consistency, facilitates cellular migration in cases of inflammation, and facilitates the healing and regeneration process of tissues [18]. Promising results of HA on physical and sexual symptoms associated with vaginal atrophy have been reported in the literature [19, 20, 21]. Most of these studies have focused on the subjective assessment of symptom response to topically applied preparations. However, HA is an endogenous molecule and it is known that its effects are best exhibited when injected into the superficial epithelial layers.

In the international patent document no WO2019003053, an application known in the state of the art, the invention demonstrates that a formulation comprising molecular oxygen and hyaluronic acid is effective in the topical treatment of vaginal disorders. This invention is used in the treatment of vaginal dryness and vulvovaginal atrophy. The oxygen used in the said invention is used as a vehicle for hyaluronic acid and also works as an enhancer of hyaluronic acid permeability. For this reason, it facilitates protein absorption by stimulating tissue regeneration as well as local microcirculation.

In the United States patent document no US20120052077, an application known in the art, the invention relates to management of vaginal health. The said application relates to pharmaceutical compositions, and methods of use thereof, for treating diseases associated with compromised boundary lubrication at the vaginal epithelium. Provided herein in certain embodiments is a method for treating a lack of vaginal lubrication or associated symptoms in an individual in need thereof, comprising topical application to the surface of the vagina, and comprising a therapeutically effective amount of PRG4 protein.

In the patent document no WO2017089475, an application known in the state of the art, the invention relates in general terms to a pharmaceutical composition for topical application, comprising an effective combination of hyaluronic acid, beta-glucan, hydrolyzed sericin, and glycerophosphoinositol or a pharmaceutically acceptable salt thereof, useful in particular as a cicatrizing agent for the treatment of micro-cracks and vulvar and/or vaginal epithelium wounds.

In the European patent document no EP1673063, an application known in the state of the art, the invention relates to relates to transdermal pharmaceutical composition consists of an estrogen and a progestin component as well as a liquid crystal gel containing polyoxyethylene-glyceryl-trioleate, propylene-glycol, isopropyl myristate and a hyaluronic acid salt or complex. The said invention is a treatment method to be used in the treatment of urinary incontinence symptoms due to urogenital atrophy, vaginal dryness, recurrent vaginitis, recurrent cystitis, painful sexual intercourse and postmenopausal estrogen deficiency.

### SUMMARY OF THE INVENTION

The objective of the invention is to obtain hyaluronic acid hydrogel comprising phytoestrogens—which are herbal substances with the same structure and functions as estradiol (Oestradiol or estradiol, an estrogenic steroid hormone) and which can produce estrogenic effects, to be used for the treatment of vaginal atrophy and vaginal rejuvenation.

Another objective of the present invention is to avoid encountering risks such as heart attack, stroke and blood clotting seen in alternative treatment methods comprising hormones.

### DETAILED DESCRIPTION OF THE INVENTION

The hydrogel formulation of the invention, which is suitable for use in vaginal tissue, is prepared in phosphate buffer or phosphate-free buffer solution and comprises
at least one of linear-linked forms of sodium hyaluronate, cross-linked forms of sodium hyaluronate, and combinations thereof,
phytoestrogens selected from a group comprising isoflavones, lignans and coumestans and combinations thereof,
vitamins and antioxidants.

In an embodiment of the invention, the invention comprises linear and/or cross-linked forms of sodium hyaluronate with a molecular weight of 100×10³ Da-3.5×10⁶ Da (determined by intrinsic viscosity values), and is present in the formulation in the concentration range of 5 mg/ml-30 mg/ml. By this means, it is ensured that the final product exhibits visco-elastic properties depending on the use of sodium hyaluronate at the specified ranges.

In an embodiment of the invention, isoflavones in the formulation are in the concentration range of 0.01 mg/ml-20 mg/ml and are selected from a group comprising geninstein, daidzein, quercetin, glycitein, formononetin, equol, o-desmethlangolensin, dihydrodaidzein, biochanin and combinations thereof.

In one embodiment of the invention, the lignans in the formulation are in the concentration range of 0.01 mg/ml-20 mg/ml and are selected from a group comprising enterodiol, enterolactone, pinoresinol, secoisolariciresinol, matairesinol, sesamin and combinations thereof

In one embodiment of the invention, the coumestans in the formulation are in the concentration range of 0.01 mg/ml-20 mg/ml and are selected from a group comprising coumestrol, wedelolactone, plicadin, and combinations thereof.

Vitamins are essential for maintaining a healthy uterus and vagina. Vitamins are involved in the metabolism of all cells and prevent tissue damage caused by oxidants. They also play an important role in balancing estrogen levels, thereby improving menopausal symptoms such as hot flashes, irritability, insomnia, dizziness, palpitations, shortness of breath, and vaginal dryness. It can be used locally on the skin as an active healing agent due to its antioxidant and anti-inflammatory properties and can reduce vaginal dryness when used topically. In an application of the invention, the vitamins in the formulation are in the concentration range of 0.05 mg/ml-30 mg/ml and are selected from a group comprising B group vitamins, namely thiamine (Vitamin B1), riboflavin (Vitamin B2), niacin (Vitamin B3), panthenol (Vitamin B5), pyridoxine (Vitamin B6); vitamin D; vitamin E; and combinations thereof.

The antioxidant addition in the formulation of the present invention neutralizes the ROS associated with vaginal inflammation that occurs during vaginal atrophy, thereby protecting the vaginal epithelium and stroma from oxidative damage. Thus, it facilitates the healthy regeneration of the lamina propria and vaginal epithelium layer. As a result, it will help the area rehydrate and expand to maintain elasticity, firmness and the proper functioning of female genital tissue. In addition, this helps to reduce the swelling that may occur after the injection. In one embodiment of the invention, the antioxidants in the formulation are selected from the group comprising glutathione, allicin, astaxanthin, N-Acetylcarnosine (NAC), epigallocatechin gallate (EGCG), coenzyme Q10 (CoQ10), alpha tocopherol, pyruvate, carotene, beta carotene, trolox, xanthan gum, hydroxytyrosol, tyrosol, caffeic acid, rutin, diosmin, melatonin, taurine, hypotaurine, vitamin C derivatives L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, sodium ascorbyl phosphate and combinations thereof.

In one embodiment of the invention, the formulation is stored in disposable sterile syringes before use.

The hyaluronic acid in the formulation of the present invention has a short half-life. In other words, it biodegrades in a short time where it is injected. Therefore, crosslinking is required to stabilize the product, and after this crosslinking reaction, the product reaches a hydrogel form. With this process, it will remain in the area where it is injected for a longer time and it will be able to maintain its moisturizing properties for several months. For this reason, the cross-linking agents to be used in cross-linking reaction in order to obtain hyaluronic acid hydrogels are selected from a group comprising 1,4-butanediol diglycidyl ether (BDDE), divinyl sulfone (DVS), 1-(3-dimethylaminopropyl)-3-ethylcarbodiimide (EDC)/N-hydroxysuccinimide (NHS) combination, glutaraldehyde, adipic acid dihydrazide and combinations thereof.

Before the sterile hydrogels to be produced within the scope of the present invention are injected, local anesthesia should be applied to the relevant area for an effective painless treatment. It is then injected very superficially into the labia majora, vulvar area, or just under the mucous tissue at the vaginal opening. Preferably 27 G½″ or 30 G½″ needles should be used during injection.

The hydrogels to be produced within the scope of the invention can be used for vaginal rejuvenation as well as the treatment of symptoms such as dryness and itching caused by vaginal atrophy. As women age, like many other parts of the body, the labia majora (the outermost labia surrounding the vagina) loses its elasticity due to tissue loss. Changes in this region may worsen after childbirth and menopause, leading to both appearance and functional concerns. The hydrogels to be produced within the scope of the invention plump up these areas that have lost elasticity and improve their appearance.

The method developed for preparing the formulation of the present invention comprises the following steps:
preparing 1% NaOH solution in deionized water,
slowly adding HA onto the said solution and dissolving it,
adding the cross-linking agent to the HA mixture and mixing it homogeneously,
incubating the mixture in a water bath at 40-50° C. for 2-5 hours, and leaving it for crosslinking reaction,
neutralizing the gel with 0.1 M hydrochloric acid (HCl) until pH becomes 6.8-7.4 in buffer solution after reaction,
filtering the mixture and washing it 5-10 times with fresh buffer solution,
reducing the gel to particle sizes of 100-500 microns (so that gels can be injected more easily from the tip of the syringe needle),
adding non-crosslinked HA to facilitate extrusion, and adding phytoestrogens and antioxidants as active ingredients to the final mixture,
adjusting the pH of the final mixture to 6.8-7.4 and filling it into the syringes under vacuum,
sterilizing the filled syringes by performing steam sterilization process,
labelling and packaging the final products.

Thanks to the hormone-free formula of the hydrogels produced within the scope of the invention, as an important advantage, they will not pose risks such as heart attack, stroke and blood clotting seen in alternative treatment methods comprising hormones. In addition, the phytoestrogens in the formulation are herbal substances that can produce estrogenic effects and have the same structure and functions as estradiol.

### REFERENCES

[1]. Dobaria, N., R. Mashru, and N. H. Vadia. “Vaginal drug delivery systems: a review of current status.” East and Central African journal of pharmaceutical sciences 10.1 (2007): 3-13.
[2]. Punitha, D. S. A. E., B. Sathya, and D. Christopher Vimalson. “Recent approaches in vaginal drug delivery systems.” International Journal of Research in Pharmacy and Pharmaceutical Sciences 3.2 (2018): 97-106.
[3]. Mac Bride, Maire B., Deborah J. Rhodes, and Lynne T. Shuster. “Vulvovaginal atrophy.” Mayo Clinic Proceedings. Vol. 85. No. 1. Elsevier, 2010.
[4]. Al-Baghdadi, O., and A. A. A. Ewies. “Topical estrogen therapy in the management of postmenopausal vaginal atrophy: an up-to-date overview.” Climacteric 12.2 (2009): 91-105.
[5]. Manson, JoAnn E., et al. “Menopausal hormone therapy and health outcomes during the intervention and extended poststopping phases of the Women's Health Initiative randomized trials.” Jama 310.13 (2013): 1353-1368.
[6]. Mitchell, Caroline M., et al. “Efficacy of vaginal estradiol or vaginal moisturizer vs placebo for treating postmenopausal vulvovaginal symptoms: a randomized clinical trial.” JAMA internal medicine 178.5 (2018): 681-690.
[7]. Buttini, Melissa J., and Christopher Maher. “The first published randomised controlled trial of laser treatment for vaginal atrophy raises serious questions.” Med J Aust 209.9 (2018): 376-377.
[8]. Acarturk, Fusun. “Mucoadhesive vaginal drug delivery systems.” Recent patents on drug delivery & formulation 3.3 (2009): 193-205.
[9]. Vermani, Kavita, and Sanjay Garg. “The scope and potential of vaginal drug delivery.” Pharmaceutical science & technology today 3.10 (2000): 359-364.
[10]. Dos Santos, Aline Martins, et al. “Recent advances in hydrogels as strategy for drug delivery intended to vaginal infections.” International Journal of Pharmaceutics (2020): 119867.
[11]. Björn, Inger, and Torbjörn Bäckström. “Drug related negative side-effects is a common reason for poor compliance in hormone replacement therapy.” Maturitas 32.2 (1999): 77-86.
[12]. Colacurci, N., et al. “Effects of soy isoflavones on menopausal neurovegetative symptoms.” Minerva ginecologica 56.5 (2004): 407-412.
[13]. Horn-Ross, Pamela L. “Phytoestrogens, body composition, and breast cancer.” Cancer Causes & Control 6.6 (1995): 567-573.
[14]. Ghazanfarpour, M., R. Sadeghi, and R. Latifnejad Roudsari. “The application of soy isoflavones for subjective symptoms and objective signs of vaginal atrophy in menopause: A systematic review of randomised controlled trials.” Journal of Obstetrics and Gynaecology 36.2 (2016): 160-171.
[15]. Ghazanfarpour, M., et al. “Red clover for treatment of hot flashes and menopausal symptoms: A systematic review and meta-analysis.” Journal of Obstetrics and Gynaecology 36.3 (2016): 301-311.
[16]. Ghazanfarpour, M., et al. “Topical administration of isoflavones for treatment of vaginal symptoms in postmenopausal women: A systematic review of randomised controlled trials.” Journal of obstetrics and gynaecology 35.8 (2015): 783-787.
[17]. Balk, Judith L., et al. “A pilot study of the effects of phytoestrogen supplementation on postmenopausal endometrium.” The Journal of the Society for Gynecologic Investigation: JSGI 9.4 (2002): 238-242.
[18]. Costantino, D., and C. Guaraldi. “Effectiveness and safety of vaginal suppositories for the treatment of the vaginal atrophy in postmenopausal women: an open, non-controlled clinical trial.” Eur Rev Med Pharmacol Sci 12.6 (2008): 411-416.
[19]. Le Donne, Maria, et al. “The effect of vaginally administered genistein in comparison with hyaluronic acid on atrophic epithelium in postmenopause.” Archives of gynecology and obstetrics 283.6 (2011): 1319-1323.
[20]. Serati, Maurizio, et al. “A comparison between vaginal estrogen and vaginal hyaluronic for the treatment of dyspareunia in women using hormonal contraceptive.” European Journal of Obstetrics & Gynecology and Reproductive Biology 191 (2015): 48-50.
[21]. Chen, Junya, et al. “Evaluation of the efficacy and safety of hyaluronic acid vaginal gel to ease vaginal dryness: a multicenter, randomized, controlled, open-label, parallel-group, clinical trial.” The journal of sexual medicine 10.6 (2013): 1575-1584.

What is claimed is:

1. A hydrogel formulation configured for use in a vaginal tissue, comprising
a buffer solution,
at least one of linear-linked forms of sodium hyaluronate and cross-linked forms of the sodium hyaluronate,
antioxidants,
phytoestrogens, wherein the phytoestrogens are at least one selected from the group consisting of isoflavones, lignans, and coumestans.

2. The hydrogel formulation according to claim 1, comprising the linear-linked forms of the sodium hyaluronate and/or the cross-linked forms of the sodium hyaluronate with a molecular weight of 100×10³ Da-3.5×10⁶ Da.

3. The hydrogel formulation according to claim 1, comprising the linear-linked forms of the sodium hyaluronate and/or the cross-linked forms of the sodium hyaluronate in a concentration range of 5 mg/ml-30 mg/ml.

4. The hydrogel formulation according to claim 1, comprising the isoflavones in a concentration range of 0.01 mg/ml-20 mg/ml.

5. The hydrogel formulation according to claim 1, comprising the isoflavones, wherein the isoflavones are at least one selected from the group consisting of geninstein, daidzein, quercetin, glycitein, formononetin, equol, o-desmethlangolensin, dihydrodaidzein, and biochanin.

6. The hydrogel formulation according to claim 1, comprising the lignans in a concentration range of 0.01 mg/ml-20 mg/ml.

7. The hydrogel formulation according to claim 1, comprising the lignans, wherein the lignans are at least one selected from the group consisting of enterodiol, enterolactone, pinoresinol, secoisolariciresinol, matairesinol, and sesamin.

8. The hydrogel formulation according to claim 1, comprising the coumestans in a concentration range of 0.01 mg/ml-20 mg/ml.

9. The hydrogel formulation according to claim 1, comprising the coumestans, wherein the coumestans are at least one selected from the group consisting of coumestrol, wedelolactone, and plicadin.

10. The hydrogel formulation according to claim 1, further comprising vitamins in a concentration range of 0.05 mg/ml-30 mg/ml.

11. The hydrogel formulation according to claim 1, further comprising vitamins, wherein the vitamins are at least one selected from the group consisting of vitamin B, vitamin D, and vitamin E, the vitamin B comprises thiamine (Vitamin B1), riboflavin (Vitamin B2), niacin (Vitamin B3), panthenol (Vitamin B5), and pyridoxine (Vitamin B6).

12. The hydrogel formulation according to claim 1, comprising the antioxidants in a concentration range of 0.05 mg/ml-30 mg/ml.

13. The hydrogel formulation according to claim 1-or 12, comprising the antioxidants, wherein the antioxidants are at least one selected from the group consisting of glutathione, allicin, astaxanthin, N-Acetylcarnosine (NAC), epigallocatechin gallate (EGCG), coenzyme Q10 (CoQ10), alpha tocopherol, pyruvate, carotene, beta carotene, trolox, hydroxytyrosol, tyrosol, caffeic acid, rutin, diosmin, melatonin, taurine, hypotaurine, vitamin C derivatives L-ascorbic acid, tetrahexyldecyl ascorbate, ascorbyl glucoside, ethylated ascorbic acid, ascorbyl palmitate, magnesium ascorbyl palmitate, magnesium ascorbyl phosphate, calcium ascorbate, sodium ascorbate, and sodium ascorbyl phosphate.

14. The hydrogel formulation according to claim 1, further comprising cross-linking agents to be used in a cross-linking reaction of hyaluronic acid hydrogels, wherein the cross-linking agents are at least one selected from the group consisting of 1,4-butanediol diglycidyl ether (BDDE), divinyl sulfone (DVS), a 1-(3-dimethylaminopropyl)-3-ethylcarbodiimide (EDC)/N-hydroxysuccinimide (NHS) combination, glutaraldehyde, and adipic acid dihydrazide.

15. The hydrogel formulation according to claim 1, wherein the hydrogel formulation is stored in disposable sterile syringes.

16. The hydrogel formulation according to claim 1, wherein the hydrogel formulation is injected superficially to a labia majora, a vulvar area, or right under a mucous tissue at a vaginal opening, and a local anesthesia is applied to the labia majora, the vulvar area, or right under the mucous tissue at the vaginal opening.

17. The hydrogel formulation according to claim 1, wherein the hydrogel formulation is used for a treatment of dryness and itching symptoms caused by vaginal atrophy, and vaginal rejuvenation.

18. The hydrogel formulation according to claim 1, wherein the hydrogel formulation is used for plumping and improving an appearance of labia majora areas losing an elasticity due to an age-related tissue loss.

19. A production method of the hydrogel formulation according to claim 1, comprising steps of
preparing a 1% NaOH solution in deionized water,
slowly adding hyaluronic acid (HA) onto the 1% NaOH solution and dissolving the HA to obtain a HA mixture,
adding a cross-linking agent to the HA mixture and mixing homogeneously to obtain a first mixture,
incubating the first mixture in a water bath at 40-50° C. for 2-5 hours, and leaving the first mixture for a crosslinking reaction to obtain a gel,
neutralizing the gel with 0.1 M hydrochloric acid (HCl) until a pH of the gel becomes 6.8-7.4 in the buffer solution after the crosslinking reaction to obtain a neutral gel,
filtering the neutral gel and washing the neutral gel 5-10 times with a fresh buffer solution,
reducing the neutral gel after washing to particle sizes of 100-500 microns to obtain a second mixture,
adding non-crosslinked HA to facilitate an extrusion, and adding the phytoestrogens and the antioxidants as active ingredients to the second mixture to obtain a third mixture,
adjusting a pH of the final third mixture to 6.8-7.4 and filling the third mixture into syringes under vacuum,
sterilizing filled syringes by performing a steam sterilization process to obtain final products,
labelling and packaging the final products.

20. The hydrogel formulation according to claim 2, comprising the linear-linked forms of the sodium hyaluronate and/or the cross-linked forms of the sodium hyaluronate in a concentration range of 5 mg/ml-30 mg/ml.
